(19) United States
(12) Patent Application Publication
(10) Pub. No.: US 2021/0261714 A1
(43) Pub. Date: Aug. 26, 2021

(54) PREPARATION OF A 2,6-DI(C1-7 ALKYL) PHENOL COMPOSITION AND A POLY(PHENYLENE ETHER)

(71) Applicants: Madhav GHANTA, Selkirk, NY (US); SHPP Global Technologies B.V., Bergen op Zoom (NL)

(72) Inventor: Madhav Ghanta, Selkirk, NY (US)

(21) Appl. No.: 17/264,111

(22) PCT Filed: Jul. 23, 2019
(86) PCT No.: PCT/US2019/042917

§ 371 Date: Jan. 28, 2021

(30) Foreign Application Priority Data
Aug. 28, 2018 (EP) 18191291.6

(51) Int. Cl. C08G 12/24; C07C 37/16; C07C 37/74; C07C 39/07

(52) U.S. Cl. CPC C08G 12/24; C07C 37/16; C07C 2601/16; C07C 39/07; C07C 37/74

(57) ABSTRACT

A process for the preparation of a 2,6-di(C₁₋₇ alkyl)phenol composition includes reacting phenol and a C₁₋₇ alkyl alcohol in the presence of a catalyst to form the 2,6-di(C₁₋₇ alkyl)phenol; and isolating the 2,6-di(C₁₋₇ alkyl)phenol composition by distillation N using a reflux ratio of greater than or equal to 4.0. The 2,6-di(C₁₋₇ alkyl)phenol composition includes less than or equal to 0.2 wt % of chain-stopper impurities, based on the total weight of the 2,6-di(C₁₋₇ alkyl)phenol composition. The 2,6-di(C₁₋₇ alkyl)phenol composition can be useful for the preparation of poly(phenylene ethers).

### BACKGROUND

Poly(phenylene ether)s constitute a family of engineering thermoplastics which are highly regarded for their chemical and physical properties. Poly(phenylene ether)s can be prepared by the oxidative coupling of a phenol with oxygen in the presence of a catalyst (e.g., a copper-amine catalyst). The reaction can be carried out in a tertiary amine, for instance, which serves as a component of the catalyst as well as a solvent for the reaction mixture. Oxygen is bubbled through the reaction mixture and an exothermic reaction takes place, producing the corresponding poly(phenylene ether).

While high molecular weight polymers have been made possible by the oxidative coupling procedure, it has been found that the molecular weight of the final polymer is not easily controlled. One reason is that the polymer molecular weight increases slowly during the early stages of the reaction, but quite rapidly during the later stages. Another reason is that the rate at which the molecular weight increases in the latter stages of reaction is dependent upon the amount of remaining active catalyst. The amount of active catalyst decreases during the reaction as a complex function of the initial reaction ingredients, the temperature history of the reaction, and the degree of oxygen dispersion in the reactor. These factors make it difficult to control the molecular weight of the polymer by stopping the reaction before its completion.

Additionally, the presence of various “chain-stopper impurities” can be detrimental to the preparation of high molecular weight poly(phenylene ether). Generally, a chain stopper is a phenol having a non-displaceable substituent in the 4-position. Examples include phenols having alkyl substituents in the 4-position, such as 4-methylphenol (p-cresol) and 2,4-dimethylphenol. It is therefore desirable to reduce the concentration of chain stoppers in order to achieve the highest possible poly(phenylene ether) molecular weight.

It would therefore be advantageous to provide monomers having low levels of chain-stopper impurities in order to provide an improved method for the preparation of high molecular weight poly(phenylene ether).

### BRIEF DESCRIPTION

A process for the preparation of a 2,6-di(C₁₋₇ alkyl)phenol composition comprises reacting phenol and a C₁₋₇ alkyl alcohol in the presence of a catalyst to form the 2,6-di(C₁₋₇ alkyl)phenol; and isolating the 2,6-di(C₁₋₇ alkyl)phenol composition by distillation using a reflux ratio of greater than or equal to 4.0; wherein the 2,6-di(C₁₋₇ alkyl)phenol composition comprises less than or equal to 0.2 wt % of chain-stopper impurities, based on the total weight of the 2,6-di(C₁₋₇ alkyl)phenol composition.

A 2,6-di(C₁₋₇ alkyl)phenol composition prepared by the process is also disclosed.

A process for the preparation of a poly(phenylene ether) comprises reacting oxygen with the 2,6-di(C₁₋₇ alkyl)phenol composition in the presence of a metal catalyst complex to form the poly(phenylene ether); and isolating the poly(phenylene ether) by precipitation; wherein the reacting is conducted in an organic solvent; the 2,6-di(C₁₋₇ alkyl)phenol composition is present in a concentration of 5 to 15 weight percent based on the total weight of the 2,6-di(C₁₋₇ alkyl)phenol and the solvent; the molar ratio of the catalyst metal to the phenol is 1:100 to 1:200; and the poly(phenylene ether) has an intrinsic viscosity of greater than 1 deciliter per gram, preferably 1.2 to 1.5 deciliter per gram, as measured in chloroform at 25° C.

The above described and other features are exemplified by the following detailed description.

### DETAILED DESCRIPTION

The present inventor has advantageously discovered a process for producing a 2,6-di(C₁₋₇ alkyl)phenol composition which includes less than or equal to 0.2 wt % of chain-stopper impurities, based on the total weight of the 2,6-di(C₁₋₇ alkyl)phenol composition. The 2,6-di(C₁₋₇ alkyl)phenol composition obtained by the process of the present disclosure can be useful for providing high molecular weight poly(phenylene ether).

Accordingly, an aspect of the present disclosure is a process for the preparation of a 2,6-di(C₁₋₇ alkyl)phenol composition. The process comprises reacting phenol and a C₁₋₇ alkyl alcohol in the presence of a catalyst to form a 2,6-di(C₁₋₇ alkyl)phenol composition, and isolating the 2,6-di(C₁₋₇ alkyl)phenol composition by distillation using a reflux ratio of greater than or equal to 4.0.

The C₁₋₇ alkyl alcohol can be a primary or secondary alcohol, preferably a primary alcohol. The C₁₋₇ alkyl alcohol can further be saturated or unsaturated, and branched or unbranched. Exemplary C₁₋₇ alkyl alcohols can include, but are not limited to, methyl alcohol, ethyl alcohol, isopropyl alcohol, n-propyl alcohol, n-butyl alcohol, isobutyl alcohol, tert-butyl alcohol, and the like, or a combination thereof. The C₁₋₇ alkyl alcohol can be a C₁₋₅ alkyl alcohol, or a C₁₋₃ alkyl alcohol, or can preferably comprise methyl alcohol (methanol). When a C₁₋₅ alkyl alcohol is used, the product is a 2,6-di(C₁₋₅ alkyl)phenol composition. When a C₁₋₃ alkyl alcohol is used, the product is a 2,6-di(C₁₋₃ alkyl)phenol composition. When the C₁₋₇ alkyl alcohol is methanol, the 2,6-di(C₁₋₇ alkyl)phenol composition comprises 2,6-xylenol (i.e., is a 2,6-xylenol composition).

The catalyst used in the alkylation reaction can be obtained by the calcination of a catalyst precursor system comprising at least one metal oxide precursor, which is converted to a metal oxide during calcination, a promoter and a pore-former. Metal oxide precursors can include magnesium oxide precursors, iron oxide precursors, chromium oxide precursors, vanadium oxide precursors, copper oxide precursors, lanthanum oxide precursors and combinations thereof. The metal oxide precursor can comprise any metal reagent which yields the corresponding metal oxide under calcination conditions such as nitrates, carbonates, oxides, hydroxides, sulfates and combinations thereof. For example, any magnesium reagent which yields magnesium oxide after calcination can be used. In one embodiment the metal oxide precursor comprises magnesium hydroxide, magnesium nitrate, magnesium carbonate, magnesium sulfate, magnesium acetate or a combination thereof. In another embodiment the metal oxide precursor comprises magnesium carbonate.

The pore former used in the catalyst system is a substance capable of aiding the formation of pores in the catalyst. Under the calcination conditions described herein the pore former decomposes or burns off leaving behind pores in the catalyst. The pore former can comprise waxes and polysaccharides. Exemplary waxes include, but are not limited to, paraffin wax, polyethylene wax, microcrystalline wax, montan wax, and combinations thereof. Exemplary polysaccharides can include cellulose, carboxyl methyl cellulose, cellulose acetate, starch, walnut powder, citric acid, polyethylene glycol, oxalic acid, stearic acid and combinations thereof. Also useful are anionic and cationic surfactants, typically long chain (C₁₀₋₂₈) hydrocarbons containing neutralized acid species, e.g., carboxylic acid, phosphoric acid, and sulfonic acid species. In an embodiment the pore former is polyethylene glycol.

The amount of the pore former employed is that which provides for average pore diameters of 100 to 400 Angstroms (Å) after calcination. The amount of pore former can be 100 ppm to 10 weight percent (wt %), or 100 ppm to 5 wt %, or up to 2 wt % with respect to catalyst precursor reagent. The pore former is typically blended with the metal oxide precursor and transition metal to provide uniform distribution of the pore former along with other components of the catalyst such as binders and fillers. Transition metal elements are used as promoters in the catalyst system. Specific examples of suitable transition metal elements can include copper, chromium, zinc, cobalt, nickel, manganese and combinations thereof. In an embodiment the promoter is copper.

The catalyst precursor system is converted to the catalyst through calcination. In some embodiments gas, such as air, nitrogen, or a combination thereof, is passed through the catalyst precursor system during all or part of the calcination. The catalyst precursor system can be heated prior to calcination and heating can also occur with gas flow. It is believed that gas flow can aid in the formation of pores having the desired pore size.

Calcination is usually carried out by heating the catalyst at a temperature sufficient to convert the metal oxide precursor to the corresponding metal oxide. Useful calcination procedures are found in U.S. Pat. Nos. 6,294,499 and 4,554,267. The calcination temperature can vary somewhat, but is usually 350 to 600° C. Slow heating rates can lead to desirable larger pore sizes but often at the expense of lower activity of the resultant catalyst. Typically, the heating rate for commercial scale will be to raise the temperature from ambient to 400° C. over a 12 to 18 hour range although the exact rate can vary depending on the actual reactor size and geometry. The calcination atmosphere can be oxidizing, inert, or reducing. Alternatively, the catalyst can be calcined at the beginning of the alkylation reaction. In other words, calcination can take place in the presence of the alkylation feed materials, i.e., the 2,6-di(C₁₋₇ alkyl)phenol composition and the alkyl alcohol. The surface area of the catalyst after calcination is usually 100 m²/g to 250 m²/g, based on grams of metal oxide.

Isolating the 2,6-di(C₁₋₇ alkyl)phenol composition is by distillation using a reflux ratio of greater than or equal to 4.0. Within this range, the reflux ratio can preferably be 4.0 to 4.5, more preferably 4.05 to 4.25.

Advantageously, the 2,6-di(C₁₋₇ alkyl)phenol composition prepared according to the method describe herein comprises less than or equal to 0.2 wt %, or less than 0.15 wt %, or less than 0.1 wt %, or less than 0.08 wt % of chain-stopper impurities, based on the total weight of the 2,6-di(C₁₋₇ alkyl)phenol composition. The chain-stopper impurities can include, but are not limited to, 2,4-di(C₁₋₇ alkyl)phenol, 4-(C₁₋₇ alkyl)phenol, 2,4,6-tri(C₁₋₇ alkyl)phenol, or a combination thereof. In a specific embodiment, when the C₁₋₇ alkyl alcohol comprises methanol, the chain-stopper impurities can comprise 2,4-xylenol, p-cresol and 2,4,6-trimethyl phenol, or a combination thereof.

In a specific embodiment, the process of the present disclosure comprises reacting phenol with methanol in the presence of a magnesium-containing catalyst comprising a copper promoter to form a 2,6-xylenol composition, and isolating the 2,6-xylenol composition by distillation using a reflux ratio of greater than or equal to 4.0, preferably 4.0 to 4.5, more preferably 4.05 to 4.25. The 2,6-xylenol composition can advantageously comprise less than or equal to 0.2 wt % of chain-stopper impurities, based on the total weight of the 2,6-xylenol composition, and the chain-stopper impurities can comprise 2,4-xylenol, p-cresol and 2,4,6-trimethyl phenol, or a combination thereof.

The 2,6-di(C₁₋₇ alkyl)phenol composition can be particularly useful for preparing poly(phenylene ether). Thus, another aspect of the present disclosure is a process for the preparation of a poly(phenylene ether). The process comprises reacting oxygen with the 2,6-di(C₁₋₇ alkyl)phenol composition obtained by the process described herein. Reacting oxygen with the 2,6-di(C₁₋₇ alkyl)phenol composition can be in the presence of a metal catalyst complex and can provide the poly(phenylene ether).

In some embodiments, the reaction is conducted in an organic solvent, the concentration of the 2,6-di(C₁₋₇ alkyl)phenol composition is 5 to 15 weight percent, or 5 to 10 weight percent, based on the total weight of the 2,6-di(C₁₋₇ alkyl)phenol composition and the solvent, and the molar ratio of the catalyst metal to the phenol is 1:100 to 1:200.

Suitable catalysts for the synthesis of poly(phenylene ether) include those comprising such catalyst metals as manganese, chromium, copper, and mixtures comprising at least one of the foregoing metals. Among metal complex catalysts, it is preferred to use a copper complex catalyst comprising a secondary alkylene diamine ligand. The copper source for the copper complex comprising a secondary alkylene diamine can comprise a salt of cupric or cuprous ion, including halides, oxides and carbonates. Alternatively, copper can be provided in the form of a pre-formed salt of the alkylene diamine ligand. Preferred copper salts include cuprous halides, cupric halides, and their mixtures. Especially preferred are cuprous bromides, cupric bromides, and their mixtures.

A preferred copper complex catalyst comprises a secondary alkylene diamine ligand. Suitable secondary alkylene diamine ligands are described in U.S. Pat. No. 4,028,341 to Hay and are represented by the formula

R^{b}—NH—R^{a}—NH—R^{c}

wherein R^{a} is a substituted or unsubstituted divalent residue wherein two or three aliphatic carbon atoms form the closest link between the two diamine nitrogen atoms; and R^{b} and R^{c} are each independently isopropyl or a substituted or unsubstituted C₄₋₈ tertiary alkyl group. Examples of R^{a} include ethylene, 1,2-propylene, 1,3-propylene, 1,2-butylene, 1,3-butylene, 2,3-butylene, the various pentylene isomers having from two to three carbon atoms separating the two free valances, phenylethylene, tolylethylene, 2-phenyl-1,2-propylene, cyclohexylethylene, 1,2-cyclohexylene, 1,3-cyclohexylene, 1,2-cyclopropylene, 1,2-cyclobutylene, 1,2-cyclopentylene, and the like. Preferably, R^{a} is ethylene. Examples of R^{b} and R can include isopropyl, t-butyl, 2-methyl-but-2-yl, 2-methyl-pent-2-yl, 3-methyl-pent-3-yl, 2,3-dimethyl-buty-2-yl, 2,3-dimethylpent-2-yl, 2,4dimethyl-pent-2-yl, 1-methylcyclopentyl, 1-methylcyclohexyl and the like. A highly preferred example of R^{b} and R^{c} is t-butyl. An exemplary secondary alkylene diamine ligand is N,N′-di-t-butylethylenediamine (DBEDA). Suitable molar ratios of copper to secondary alkylene diamine are from 1:1 to 1:5, preferably 1:1 to 1:3, more preferably 1:1.5 to 1:2.

The preferred copper complex catalyst comprising a secondary alkylene diamine ligand can further comprise a secondary monoamine. Suitable secondary monoamine ligands are described in commonly assigned U.S. Pat. No. 4,092,294 to Bennett et al. and represented by the formula

R^{d}—NH—R^{e}

wherein R^{d} and R^{e} are each independently substituted or unsubstituted C₁₋₁₂ alkyl groups, and preferably substituted or unsubstituted C₃₋₆ alkyl groups. Examples of the secondary monoamine include di-n-propylamine, di-isopropylamine, di-n-butylamine, di-sec-butylamine, di-t-butylamine, N-isopropyl-t-butylamine, N-sec-butyl-t-butylamine, di-n-pentylamine, bis(1,1-dimethylpropyl)amine, and the like. A highly preferred secondary monoamine is di-n-butylamine (DBA). A suitable molar ratio of copper to secondary monoamine is from 1:1 to 1:10, preferably 1:3 to 1:8, and more preferably 1:4 to 1:7.

The preferred copper complex catalyst comprising a secondary alkylene diamine ligand can further comprise a tertiary monoamine. Suitable tertiary monoamine ligands are described in the abovementioned Hay U.S. Pat. No. 4,028,341 and Bennett U.S. Pat. No. 4,092,294 patents and include heterocyclic amines and certain trialkyl amines characterized by having the amine nitrogen attached to at least two groups which have a small cross-sectional area. In the case of trialkylamines, it is preferred that at least two of the alkyl groups be methyl with the third being a primary C₁₋₈ alkyl group or a secondary C₃₋₈ alkyl group. It is especially preferred that the third substituent have no more than four carbon atoms. A highly preferred tertiary amine is dimethylbutylamine (DMBA). A suitable molar ratio of copper to tertiary amine is less than 1:20, preferably less than 1:19, more preferably 1:1 to less than 1:19, more preferably 1:15 to less than 1:19, even more preferable 1:16 to less than 1:19.

A suitable molar ratio of metal complex catalyst (measured as moles of metal) to 2,6-di(C₁₋₇ alkyl)phenol composition is 1:50 to 1:400, preferably 1:100 to 1:200, more preferably 1:100 to 1:180.

The reaction between oxygen and the 2,6-di(C₁₋₇ alkyl)phenol composition conducted in the presence of a metal complex catalyst can optionally be conducted in the presence of bromide ion. It has already been mentioned that bromide ion can be supplied as a cuprous bromide or cupric bromide salt. Bromide ion can also be supplied by addition of a 4-bromophenol, such as 2,6-dimethyl-4-bromophenol. Additional bromide ion can be supplied in the form of hydrobromic acid, an alkali metal bromide, or an alkaline earth metal bromide. Sodium bromide and hydrobromic acid are highly preferred bromide sources. A suitable ratio of bromide ion to copper ion is 2 to 20, preferably 3 to 20, more preferably 4 to 7.

In the process, the reaction between oxygen and the 2,6-di(C₁₋₇ alkyl)phenol composition is carried out in an organic solvent. Suitable organic solvents include alcohols, ketones, aliphatic and aromatic hydrocarbons, chlorohydrocarbons, nitrohydrocarbons, ethers, esters, amides, mixed ether-esters, sulfoxides, and the like, providing they do not interfere with or enter into the oxidation reaction. The very high molecular weight poly(phenylene ethers) can greatly increase the viscosity of the reaction mixture. Therefore, it is sometimes desirable to use a solvent system that will cause them to precipitate while permitting the lower molecular weight polymers to remain in solution until they form the higher molecular weight polymers. Preferred solvents include aromatic hydrocarbons. In some embodiments, the organic solvent comprises toluene.

A suitable starting concentration of phenol is 5 to 15 weight percent, preferably 5 to 10 weight percent, more preferably 6 to 10 weight percent, based on the total weight of 2,6-di(C₁₋₇ alkyl)phenol composition and solvent. All the 2,6-di(C₁₋₇ alkyl)phenol composition can be added at the beginning of the reaction. Alternatively, the 2,6-di(C₁₋₇ alkyl)phenol composition can be added in discrete or continuous amounts during the course of the reaction. Oxygen can be introduced into reaction mixture in pure form or diluted with an inert gas such as nitrogen, helium, argon, and the like. Air can be used as an oxygen source. It is contemplated to control the 2,6-di(C₁₋₇ alkyl)phenol composition and oxygen addition rates as described in European Patent Application No. 107,653 A1 so that about ¼ to ¾ of the 2,6-di(C₁₋₇ alkyl)phenol composition is in prepolymer state when about/3 to ⅔ of the charge of 2,6-di(C₁₋₇ alkyl)phenol composition has been added to the reaction mixture.

The reaction between 2,6-di(C₁₋₇ alkyl)phenol composition and oxygen can optionally be carried out in the presence of one or more additional components, including a lower alkanol or glycol, a small amount of water, or a phase transfer agent. It is generally not necessary to remove reaction byproduct water during the course of the reaction.

The polymerization reaction can be carried out at an initial temperature of 25 to 35° C., with heating from 40 to 50° C. after the exothermic portion of the reaction profile. There is no particular limitation on the way the reaction is monitored or terminated. As the reaction proceeds, the increase in the product poly(phenylene ether) intrinsic viscosity can be continuously followed by an in-line viscometer. The reaction can be terminated by stopping the oxygen addition when the target intrinsic viscosity is reached. Other suitable methods for terminating the reaction include the addition of a mineral or organic acid, such as acetic acid, or the addition of a sequestrant as described in greater detail below.

There is no particular limitation on the apparatus used to carry out the poly(phenylene ether) preparation. The reaction can be carried out both in the batch, semi-batch, or continuous modes. Programmed addition of portions of the 2,6-di(C₁₋₇ alkyl)phenol composition at various points in the reaction can be employed. Various types of reactors can be used for the polymerization, including a single stirred tank reactor, two or more continuous stirred tank reactors in series, a bubble column reactor, or a column reactor. In order to minimize formation of byproduct tetramethyldiphenoquinone (TMDQ) and achieve a narrow molecular weight distribution and short reaction times, it can be preferred to use a semi-batch reactor, two or more continuous stirred tank reactors in series, or a plug-flow bubble column reactor.

The method can further comprise recovering the copper catalyst using an aqueous sequestrant solution. Suitable techniques for recovering the catalyst metal from the metal complex catalyst include those described in commonly assigned U.S. Pat. No. 3,838,102 to Bennett et al., U.S. Pat. No. 3,951,917 to Floryan et al., and U.S. Pat. No. 4,039,510 to Cooper et al. These techniques comprise the addition of one or more sequestrants to complex the catalyst metal and facilitate its separation from the poly(phenylene ether) product. A preferred method for removing catalyst metal from the poly(phenylene ether) product is described in U.S. application Ser. No. 09/616,737. This method, which eliminates multiple rinses with a complexing reagent, includes removing the catalyst from the polymerization mixture by mixing the polymerization mixture with a complexing reagent and liquid/liquid centrifuging the multiphase mixture. Water is then added to the polymer phase prior to a subsequent liquid/liquid centrifuge process. In general, suitable sequestrants include polyfunctional carboxylic acid-containing compounds, such as citric acid, tartaric acid, nitrilotriacetic acid, ethylenediaminetetraacetic acid, ethylenediaminedisuccinic acid, hydroxyethylethylenediaminetriacetic acid, diethylenetriaminepentaacetic acid, and the like. These sequestrants can be used as their free acids or salts of, for example, their alkali metals, alkaline earth metals, and nitrogenous cations. Preferred sequestrants include nitrilotriacetic acid, ethylenediamine tetraacetic acid and their salts. Suitable molar ratios of sequestrant to catalyst metal are 1:1 to 5:1, preferably 1.1:1 to 3:1, more preferably 1:1.5 to 1:2.5.

The method further comprises isolating the poly(phenylene ether) by precipitation. Precipitation of the poly(phenylene ether) can be induced by appropriate selection of reaction solvent described above, or by the addition of an anti-solvent to the reaction mixture. Suitable anti-solvents include lower alkanols having one to about ten carbon atoms, acetone and hexane. The preferred anti-solvent is methanol. The anti-solvent can be employed at a range of concentrations relative to the organic solvent, with the optimum concentration depending on the identities of the organic solvent and anti-solvent, as well as the concentration and intrinsic viscosity of the poly(phenylene ether) product. It has been discovered that when the organic solvent is toluene and the anti-solvent is methanol, a toluene:methanol weight ratio of 50:50 to 80:20 is suitable, with ratios of 60:40 to 70:30 being preferred, and 63:37 to 67:33 being more preferred. These preferred and more preferred ratios are useful for producing a desirable powder morphology for the isolated poly(phenylene ether) resin, without generating either stringy powder or excessive powder fines.

The isolated poly(phenylene ether) can have an intrinsic viscosity of greater than 1 deciliter per gram, preferably 1.2 to 1.5 deciliter per gram, as measured in chloroform at 25° C. using an Ubbelohde viscometer.

Thus, the poly(phenylene ether) prepared according to the above method represents another aspect of the present disclosure. The poly(phenylene ether) can comprise repeating structural units having the formula

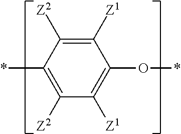

wherein each occurrence of Z¹ is independently halogen, unsubstituted or substituted C₁₋₁₂ hydrocarbyl provided that the hydrocarbyl group is not tertiary hydrocarbyl, C₁₋₁₂ hydrocarbylthio, C₁₋₁₂ hydrocarbyloxy, or C₂₋₁₂ halohydrocarbyloxy wherein at least two carbon atoms separate the halogen and oxygen atoms; and each occurrence of Z² is independently hydrogen, halogen, unsubstituted or substituted C₁₋₁₂ hydrocarbyl provided that the hydrocarbyl group is not tertiary hydrocarbyl, C₁₋₁₂ hydrocarbylthio, C₁₋₁₂ hydrocarbyloxy, or C₂₋₁₂ halohydrocarbyloxy wherein at least two carbon atoms separate the halogen and oxygen atoms. As used herein, the term “hydrocarbyl”, whether used by itself, or as a prefix, suffix, or fragment of another term, refers to a residue that contains only carbon and hydrogen. The residue can be aliphatic or aromatic, straight-chain, cyclic, bicyclic, branched, saturated, or unsaturated. It can also contain combinations of aliphatic, aromatic, straight chain, cyclic, bicyclic, branched, saturated, and unsaturated hydrocarbon moieties. However, when the hydrocarbyl residue is described as substituted, it may, optionally, contain heteroatoms over and above the carbon and hydrogen members of the substituent residue. Thus, when specifically described as substituted, the hydrocarbyl residue can also contain one or more carbonyl groups, amino groups, hydroxyl groups, or the like, or it can contain heteroatoms within the backbone of the hydrocarbyl residue. As one example, Z¹ can be a di-n-butylaminomethyl group formed by reaction of a terminal 3,5-dimethyl-1,4-phenyl group with the di-n-butylamine component of an oxidative polymerization catalyst.

The poly(phenylene ether) can comprise molecules having aminoalkyl-containing end group(s), typically located in a position ortho to the hydroxy group. Also frequently present are tetramethyldiphenoquinone (TMDQ) end groups, typically obtained from 2,6-dimethylphenol-containing reaction mixtures in which tetramethyldiphenoquinone by-product is present. The poly(phenylene ether) can be in the form of a homopolymer, a copolymer, a graft copolymer, an ionomer, or a block copolymer, as well as combinations thereof.

Articles comprising the poly(phenylene ether) made by the above method represent another aspect of the present disclosure. For example, poly(phenylene ether) having a high intrinsic viscosity can be particularly well-suited for fabrication of asymmetric hollow fiber membranes for gas separation. The poly(phenylene ether) produced by the process is also suitable for, for example, the manufacturing of fibers, films and sheets.

Accordingly, the present inventors have discovered that phenolic monomers containing a certain level of chain-stopper impurities can be suitable for the preparation of high intrinsic viscosity poly(phenylene ether). Such monomers can advantageously be prepared using a reflux ratio of greater than 4.0.

This disclosure is further illustrated by the following examples, which are non-limiting.

### EXAMPLES

2,6-Xylenol is typically produced by the vapor phase alkylation of phenol with methanol in presence of MgCO₃-based catalyst. The typical catalyst composition involves dry blending a mixture of 95.25% MgCO₃+0.25% CuNO₃+2% PPO+0.50% graphite+2% poly(ethylene glycol). The blend is tableted and the tablets are charged into the tubes of the alkylation reactor. The methanol and phenol are charged into the reactor at 2721 kg/h and 3175 kg/h, respectively, which reflects a methanol to phenol molar ratio of 2.5. The raw materials are preheated to 710° F. and vapors are fed into the reactor when the catalyst is fresh. The analysis of the reactor effluent stream during steady operation reveals the selectivity towards 2,6-xylenol, o-cresol, p-cresol, 2,4-dimethylphenol, 2,4,6-trimethylphenol to be 75%, 20%, 0.03%, 0.2% and 0.2%, respectively. During startup the selectivity towards 2,4,6-trimethylphenol is 20% and this concentration drops drastically during the first few hours of operation. The typical catalyst life is 1600 h. As the catalyst deactivates during the course of operation, the process productivity (i.e., 2,6-xylenol selectivity) is sustained by gradually increasing the reactor temperature to 880° F. (from 710° F. during startup), following which, the reactor pressure is gradually increased to 50 psig (from 15-20 psig during start-up). Lastly, the phenol feed rate to the reactor is increased from 3175 kg/h to 3400 kg/h, to sustain productivity.

Improvements in the purity of the monomer 2,6-xylenol were pursued by increasing the reflux ratio during purification of the monomer at a constant temperature. Over a period of 6 hours, the reflux ratio was increased from 3.59 to 4.17. Table 1 shows the concentrations of various chain stoppers at the varying reflux ratios over the course of the 6 hours. Chain stopper concentration was measured by gas chromatography. The amount of each chain stopper is given in weight percent, based on the total weight of the 2,6-xylenol mixture.

TABLE 1

| Sample | 1 | 2 | 3 | 4 | 5 | 6 | 7 |
|---|---|---|---|---|---|---|---|
| Conditions | RR = 3.59 | RR = 4.13 | RR = 4.09 | RR = 4.10 | RR = 4.10 | RR = 4.17 | RR = 4.17 |
| Production Rate, lb/h | 3002.00 | 3000.00 | 3000.00 | 2999.00 | 2997.00 | 3449.00 | 3501.00 |
| o-cresol | 0.0495 | 0.04544 | 0.03880 | 0.04052 | 0.03202 | 0.04434 | 0.02582 |
| p-cresol | 0.00286 | 0.00204 | 0.00243 | 0.00230 | 0.00054 | 0.00170 | 0.00053 |
| Dibenzofuran | 0.00402 | 0.00262 | 0.00384 | 0.00329 | 0.00040 | 0.00174 | 0.00051 |
| 2,6-xylenol | 99.89 | 99.91 | 99.92 | 99.92 | 99.95 | 99.93 | 99.95 |
| 2,4-xylenol | 0.00702 | 0.00320 | 0.00125 | 0.00098 | 0 | 0 | 0 |
| Mesitol | 0.00147 | 0 | 0 | 0 | 0 | 0 | 0 |

Table 2 shows quantitative gas chromatography analysis of 2,6-xylenol prepared using a reflux ratio of 3.59 compared to 2,6-xylenol prepared using a reflux ratio of 4.10.

TABLE 2

| Components | 8 | 9 |
|---|---|---|
| Reflux Ratio | 4.10 | 3.59 |
| Phenol | 0.00104 | 0.000583 |
| Dimethylcyclohexane | 0.0165 | 0.00777 |
| o-cresol | 0.0245 | 0.007756 |
| p-cresol | 0.0061 | 0.01017 |
| Dibenzofuran | 0.0075 | 0.0057 |
| 2,6-xylenol | 99.91 | 99.74 |
| o-E Phenol | 0.008784 | 0.02169 |
| 2,4-xylenol | 0.00357 | 0.08157 |
| 2,4,6-trimethyl anisole | 0.000748 | 0.00215 |
| 7-methyl dibenzofuran | — | 0.049 |
| 2,6-ethyl methyl phenol | — | 0.0531 |
| Mesitol | — | 0.0108 |

As shown in Table 2, when the reflux ratio is increased from 3.59 to 4.10, the concentration of chain stoppers such as p-cresol and 2,4-xylenol is reduced from 0.01017 wt % to 0.0061 wt. % and 0.08157 wt. % to 0.00357 wt. %, respectively.

The collected monomer samples was polymerized for making high intrinsic viscosity (IV) poly(phenylene ether) (PPE). Materials used for the polymerization as described in Table 3.

TABLE 3

| Component | Description |
|---|---|
| DMP | 2,6-Dimethylphenol (2,6-xylenol), CAS Reg. No. |
| | 576-26-1, 99.9% pure; obtained from ACROS |
| | Organics or Deepak Novochem Ltd., or produced |
| | according to the above procedure. |
| Cu₂O | Cuprous oxide, CAS Reg. No. 1317-39-1; obtained |
| | from American Chemet. |
| HBr | Hydrobromic acid, CAS Reg. No. 10035-10-6; |
| | obtained as a 48 weight percent solution in water |
| | from Chemtura. |
| DBEDA | N,N'-di-tert-butylethylenediamine, CAS Reg. No. |
| | 4062-60-6; obtained from Achiewell. |
| DBA | N,N-di-n-butylamine, CAS Reg. No. 111-92-2; |
| | obtained from Tampico. |
| DMBA | N,N-dimethylbutylamine, CAS Reg. No. 927-62-8; |
| | obtained from Oxea. |
| QUAT | N,N,N',N'-Didecyldimethyl ammonium chloride, |
| | CAS Reg. No. 7173-51-5; obtained from Pilot |
| | Chemical. |
| NTA | Nitrolotriacetic acid trisodium salt, CAS Reg. No. |
| | 5064-31-3; obtained from Ascend. |
| Toluene | Toluene, CAS Reg. No. 108-33-3; obtained from |
| | Acros Organics. |

PPE was produced using a one gallon stainless steel reaction vessel. The total solids loading was 7 wt. %. The solids loading and percent solids refers to the weight percent of DMP based on the total weight of DMP and toluene. Approximately 10% of the total toluene solution of monomer was present in the reactor at the beginning of the reaction with the remaining solution added to the reactor over the course of 45 minutes. The copper solution was prepared by dissolving Cu₂O (0.37 grams, 0.005 moles copper ion) in a 46 wt. % aqueous solution of hydrobromic acid (3.38 grams, 0.28 moles bromide ion). The DBA loading was 4.05 wt. % based on total monomer weight (3.18 grams). The DMBA loading was 1 wt. % based on total monomer weight (9.53 grams). The DBEDA loading was 30 wt. % based on the weight of the copper ion solution. The QUAT loading was 5 wt. % based on the weight of the copper ion solution. Molecular oxygen was sparged into the reaction mixture via dip tube at 8.20 standard liters per hour (0.29 standard cubic feet per hour (SCFH); oxygen and DMP were added to the reaction mixture in a constant mole ratio of 1:1). Throughout the reaction, nitrogen (24.5 standard liters per hour; 0.86 SCFH) was added to the headspace to reduce the oxygen concentration in the gas phase. The reaction was gradually heated from room temperature (23° C.) during the exothermic stage. During the build stage, the temperature was gradually heated from 23 to 48° C. Copper ion was chelated with trisodium nitrilotriacetate at the end of the build phase, terminating the oxidative polymerization reaction. The reaction mixture was transferred to a jacketed glass vessel and allowed to equilibrate for 185 minutes. The temperature of the mixture during the equilibration phase was 62° C.

The intrinsic viscosity (IV) of the final PPE powder was measured by Ubbelohde viscometer and number average and weight average molecular weights were determined using gel permeation chromatography (GPC). Examples 10-20, shown in Table 4, summarize the results of these experiments undertaken with monomer produced according to the above procedure. Examples 21 and 22 (Table 4) are comparative examples of polymer produced with 2,6-xylenol sourced from commercial suppliers.

TABLE 4

| | | Concentration of impurity accompanying the DMP | | | | | | |
|---|---|---|---|---|---|---|---|---|
| | % | % Dimethyl | % o- | % p- | % Di- | % 2,6 | % 2-ethyl | % 2,4 Dimethyl |
| Ex. | phenol | cyclohexane | cresol | cresol | benzofuran | xylenol | phenol | phenol |
| 10 | 0.00123 | 0.01777 | 0.01234 | 0.02086 | 0.00672 | 99.79 | 0.0252 | 0.0449 |
| 11 | 0.000587 | 0.008736 | 0.007464 | 0.01089 | 0.006285 | 99.76 | 0.02094 | 0.07894 |
| 12 | 0.000516 | 0.00818 | 0.00747 | 0.01013 | 0.00575 | 99.75 | 0.0212 | 0.0804 |
| 13 | 0.000583 | 0.007777 | 0.007756 | 0.0101 | 0.0057 | 99.74 | 0.0216 | 0.0815 |
| 14 | 0.000507 | 0.005591 | 0.00952 | 0.01703 | 0.005805 | 99.82 | 0.05348 | 0.00288 |
| 15 | 0.000487 | 0.005815 | 0.009503 | 0.016882 | 0.005805 | 99.8 | 0.02044 | 0.03288 |
| 16 | 0.000536 | 0.00705 | 0.00921 | 0.01633 | 0.00705 | 99.78 | 0.02096 | 0.0693 |
| 17 | 0.000453 | 0.00786 | 0.00816 | 0.01348 | 0.007 | 99.77 | 0.02005 | 0.0745 |
| 18 | 0.000463 | 0.00845 | 0.00795 | 0.01166 | 0.006659 | 99.77 | 0.0195 | 0.076 |
| 19 | 0 | 0.00196 | 0.0063 | 0.00573 | 0.00143 | 99.91 | 0.00862 | 0.0273 |
| 20 | 0.00049 | 0.00894 | 0.007607 | 0.01149 | 0.006715 | 99.76 | 0.02 | 0.07759 |
| 21 | 0 | 0 | 0 | 0 | 0 | 99.9 | 0 | 0.00808 |
| 22 | 0 | 0 | 0 | 0.00593 | 0 | 99.83 | | 0.0518 |
| | Concentration of impurity accompanying the DMP | | | | | | | |
| | % 2,4,6 | %7 | % 2,6 ethyl | % 2,4,6 | Polymer Characterization Data | | | |
| | Trimethyl | Methyl | methyl | Trimethyl | IV, | Mn, | Mw, | |
| Ex. | anisole | dibenzofuran | phenol | phenol | dL/g | g/mol | g/mol | Dist. |
| 10 | 0.00582 | 0.04124 | 0.01854 | 0.00493 | 1.1 | 45813 | 195870 | Unimodal |
| 11 | 0.00197 | 0.041479 | 0.05212 | 0.009551 | 1.04 | 43355 | 193930 | Unimodal |
| 12 | 0.00227 | 0.0464 | 0.0528 | 0.01049 | 0.81 | 34904 | 125850 | Unimodal |
| 13 | 0.00215 | 0.049 | 0.05314 | 0.01088 | 0.95 | 43700 | 159000 | Unimodal |
| 14 | 0.002887 | 0.02287 | 0.030162 | 0.00621 | 0.91 | 36754 | 142050 | Unimodal |
| 15 | 0.00239 | 0.02836 | 0.03537 | 0.00735 | 0.89 | 43300 | 128900 | Unimodal |
| 16 | 0.0022 | 0.03175 | 0.04187 | 0.008365 | 0.88 | 44498 | 130120 | Unimodal |
| 17 | 0.002 | 0.03365 | 0.04859 | 0.0083 | 1.16 | 47294 | 203850 | Unimodal |
| 18 | 0.00203 | 0.03533 | 0.05119 | 0.0083 | 1.13 | 39530 | 344220 | Unimodal |
| 19 | 0.00055 | 0.00731 | 0.01923 | 0.00191 | 1.12 | 33943 | 180280 | Unimodal |
| 20 | 0.00193 | 0.03841 | 0.05124 | 0.008839 | 1.05 | 44581 | 226210 | Bimodal |
| 21 | 0 | 0 | 0 | 0 | 1.57 | 24397 | 264440 | Unimodal |
| 22 | 0 | 0 | 0 | 0 | 1.65 | 28219 | 259020 | Unimodal |

Poly(phenylene ether)s were prepared from monomer isolated using a reflux ratio of 4.10. The polymer was analyzed at various polymerization times. The results are summarized in Table 5. The oxygen to monomer molar ratio was 1:1 and the polymerization was conducted at room temperature (i.e., 25° C.).

TABLE 5

| | | | GPC- | | | | | wt % | wt % | | |
|---|---|---|---|---|---|---|---|---|---|---|---|
| Sam. | Time, | IV, | IV, | | | | wt % | Term. | Inter. | wt % | wt % |
| No. | min | dl/g | dl/g | Mw | Mn | D | Biphenyl | DBA | DBA | MBA | TMDQ |
| 1 | 75 | | | | | | | | | | 0.0835 |
| 2 | 135 | 1.265 | 0.997 | 221595 | 66486 | 3.33 | 0.062 | 0.146 | 0.88 | 0.084 | 0.0864 |
| 3 | 265 | 1.264 | 0.976 | 216540 | 52510 | 4.12 | 0.149 | 0.182 | 0.87 | 0.112 | 0.0595 |
| 4 | 400 | 1.106 | 0.843 | 180280 | 33943 | 5.31 | 0.443 | 0.252 | 0.76 | 0.142 | 0.0387 |

This disclosure further encompasses the following aspects.

Aspect 1: A process for the preparation of a 2,6-di(C₁₋₇ alkyl)phenol composition, the process comprising: reacting phenol and a C₁₋₇ alkyl alcohol in the presence of a catalyst to form the 2,6-di(C₁₋₇ alkyl)phenol; and isolating the 2,6-di(C₁₋₇ alkyl)phenol composition by distillation using a reflux ratio of greater than or equal to 4.0; wherein the 2,6-di(C₁₋₇ alkyl)phenol composition comprises less than or equal to 0.2 wt % of chain-stopper impurities, based on the total weight of the 2,6-di(C₁₋₇ alkyl)phenol composition.

Aspect 2: The process of aspect 1, wherein the 2,6-di(C₁₋₇ alkyl)phenol composition comprises 2,6-xylenol.

Aspect 3: The process of aspect 1 or 2, wherein the C₁₋₇ alkyl alcohol comprises methanol.

Aspect 4: The process of any one or more of aspects 1 to 3, wherein the chain-stopper impurities comprise 2,4-di(C₁₋₇ alkyl)phenol, 4-(C₁₋₇ alkyl)phenol, 2,4,6-tri(C₁₋₇ alkyl)phenol, or a combination thereof.

Aspect 5: The process of any one or more of aspects 1 to 4, wherein the chain-stopper impurities comprise 2,4-xylenol, p-cresol and 2,4,6-trimethyl phenol, or a combination thereof.

Aspect 6: The process of any one or more of aspects 1 to 5, wherein the reflux ratio is 4.0 to 4.5, preferably 4.05 to 4.25.

Aspect 7: A 2,6-di(C₁₋₇ alkyl)phenol composition prepared by the process of any one or more of aspects 1 to 6.

Aspect 8: The process according to any one or more of aspects 1 to 6, the process further comprising: reacting oxygen with the 2,6-di(C₁₋₇ alkyl)phenol composition in the presence of a metal catalyst complex to form a poly(phenylene ether); and isolating the poly(phenylene ether) by precipitation; wherein the reacting is conducted in an organic solvent; the 2,6-di(C₁₋₇ alkyl)phenol composition is present in a concentration of 5 to 15 weight percent based on the total weight of the 2,6-di(C₁₋₇ alkyl)phenol and the solvent; the molar ratio of the catalyst metal to the phenol is 1:100 to 1:200; and the poly(phenylene ether) has an intrinsic viscosity of greater than 1 deciliter per gram, preferably 1.2 to 1.5 deciliter per gram, as measured in chloroform at 25° C.

Aspect 9: The process of aspect 8, wherein the 2,6-di(C₁₋₇ alkyl)phenol composition comprises 2,6-xylenol.

Aspect 10: The process of aspect 8 or 9, wherein the chain-stopper impurities comprise 2,4-xylenol, p-cresol and 2,4,6-trimethyl phenol, or a combination thereof.

Aspect 11: A poly(phenylene ether) made by the process of any of aspects 8 to 10.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group.

As used herein, the term “hydrocarbyl”, whether used by itself, or as a prefix, suffix, or fragment of another term, refers to a residue that contains only carbon and hydrogen. The residue can be aliphatic or aromatic, straight-chain, cyclic, bicyclic, branched, saturated, or unsaturated. It can also contain combinations of aliphatic, aromatic, straight chain, cyclic, bicyclic, branched, saturated, and unsaturated hydrocarbon moieties. However, when the hydrocarbyl residue is described as substituted, it may, optionally, contain heteroatoms over and above the carbon and hydrogen members of the substituent residue. Thus, when specifically described as substituted, the hydrocarbyl residue can also contain one or more carbonyl groups, amino groups, hydroxyl groups, or the like, or it can contain heteroatoms within the backbone of the hydrocarbyl residue. The term “alkyl” means a branched or straight chain, unsaturated aliphatic hydrocarbon group, e.g., methyl, ethyl, n-propyl, i-propyl, n-butyl, s-butyl, t-butyl, n-pentyl, s-pentyl, and n- and s-hexyl. “Alkenyl” means a straight or branched chain, monovalent hydrocarbon group having at least one carbon-carbon double bond (e.g., ethenyl (—HC═CH₂)). “Alkoxy” means an alkyl group that is linked via an oxygen (i.e., alkyl-O—), for example methoxy, ethoxy, and sec-butyloxy groups. “Alkylene” means a straight or branched chain, saturated, divalent aliphatic hydrocarbon group (e.g., methylene (—CH₂—) or, propylene (—(CH₂)₃—)). “Cycloalkylene” means a divalent cyclic alkylene group, —CₙH₂ₙ₋ₓ, wherein x is the number of hydrogens replaced by cyclization(s). “Cycloalkenyl” means a monovalent group having one or more rings and one or more carbon-carbon double bonds in the ring, wherein all ring members are carbon (e.g., cyclopentyl and cyclohexyl). “Aryl” means an aromatic hydrocarbon group containing the specified number of carbon atoms, such as phenyl, tropone, indanyl, or naphthyl. “Arylene” means a divalent aryl group. “Alkylarylene” means an arylene group substituted with an alkyl group. “Arylalkylene” means an alkylene group substituted with an aryl group (e.g., benzyl). The prefix “halo” means a group or compound including one more of a fluoro, chloro, bromo, or iodo substituent. A combination of different halo groups (e.g., bromo and fluoro), or only chloro groups can be present. The prefix “hetero” means that the compound or group includes at least one ring member that is a heteroatom (e.g., 1, 2, or 3 heteroatom(s)), wherein the heteroatom(s) is each independently N, O, S, Si, or P. “Substituted” means that the compound or group is substituted with at least one (e.g., 1, 2, 3, or 4) substituents that can each independently be a C₁₋₉ alkoxy, a C₁₋₉ haloalkoxy, a nitro (—NO₂), a cyano (—CN), a C₁₋₆ alkyl sulfonyl (—S(═O)₂-alkyl), a C₆₋₁₂ aryl sulfonyl (—S(═O)₂-aryl)a thiol (—SH), a thiocyano (—SCN), a tosyl (CH₃C₆H₄SO₂—), a C₃₋₁₂ cycloalkyl, a C₂₋₁₂ alkenyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom's normal valence is not exceeded. The number of carbon atoms indicated in a group is exclusive of any substituents. For example —CH₂CH₂CN is a C₂ alkyl group substituted with a nitrile.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A process for the preparation of a 2,6-di(C₁₋₇ alkyl)phenol composition, the process comprising:
reacting phenol and a C₁₋₇ alkyl alcohol in the presence of a catalyst to form the 2,6-di(C₁₋₇ alkyl)phenol; and
isolating the 2,6-di(C₁₋₇ alkyl)phenol composition by distillation using a reflux ratio of greater than or equal to 4.0;
wherein the 2,6-di(C₁₋₇ alkyl)phenol composition comprises less than or equal to 0.2 wt % of chain-stopper impurities, based on the total weight of the 2,6-di(C₁₋₇ alkyl)phenol composition.

2. The process of claim 1, wherein the 2,6-di(C₁₋₇ alkyl)phenol composition comprises 2,6-xylenol.

3. The process of claim 1, wherein the C₁₋₇ alkyl alcohol comprises methanol.

4. The process of claim 1, wherein the chain-stopper impurities comprise 2,4-di(C₁₋₇ alkyl)phenol, 4-(C₁₋₇ alkyl)phenol, 2,4,6-tri(C₁₋₇ alkyl)phenol, or a combination thereof.

5. The process of claim 1, wherein the chain-stopper impurities comprise 2,4-xylenol, p-cresol and 2,4,6-trimethyl phenol, or a combination thereof.

6. The process of claim 1, wherein the reflux ratio is 4.0 to 4.5.

7. A 2,6-di(C₁₋₇ alkyl)phenol composition prepared by the process of claim 1.

8. The process according to claim 1, the process further comprising:
reacting oxygen with the 2,6-di(C₁₋₇ alkyl)phenol composition in the presence of a metal catalyst complex to form a poly(phenylene ether); and
isolating the poly(phenylene ether) by precipitation;
wherein
the reacting is conducted in an organic solvent;
the 2,6-di(C₁₋₇ alkyl)phenol composition is present in a concentration of 5 to 15 weight percent based on the total weight of the 2,6-di(C₁₋₇ alkyl)phenol and the solvent;
the molar ratio of the catalyst metal to the phenol is 1:100 to 1:200; and
the poly(phenylene ether) has an intrinsic viscosity of greater than 1 deciliter per gram, preferably 1.2 to 1.5 deciliter per gram, as measured in chloroform at 25° C.

9. The process of claim 8, wherein the 2,6-di(C₁₋₇ alkyl)phenol composition comprises 2,6-xylenol.

10. The process of claim 8, wherein the chain-stopper impurities comprise 2,4-xylenol, p-cresol and 2,4,6-trimethyl phenol, or a combination thereof.

11. A poly(phenylene ether) made by the process of claim 8.
